(19) United States
(12) Patent Application Publication
(10) Pub. No.: US 2013/0116505 A1
(43) Pub. Date: May 9, 2013

(54) ELECTRODE ARRANGEMENT

(71) Applicant: Wolfgang Seidel, Berg (DE)

(72) Inventor: Wolfgang Seidel, Berg (DE)

(21) Appl. No.: 13/808,518

(22) PCT Filed: Jul. 5, 2011
(86) PCT No.: PCT/EP11/03341

§ 371 Date: Jan. 4, 2013

(30) Foreign Application Priority Data
Jul. 6, 2010 (DE) 10 2010 026 210.2

(51) Int. Cl. A61B 18/14; A61B 1/018

(52) U.S. Cl. CPC A61B 18/1482; A61B 1/018
USPC 600/106; 606/49

(57) ABSTRACT

A device for coagulating body tissue contains an electrode arrangement comprising a first electrode group and a second electrode group, both of which have strip-shaped electrodes.

The invention relates to an electrode arrangement for coagulating body tissue and/or body vessels.

In radio-frequency technology, an electrical radio-frequency field is applied to an electrode to heat and/or shrink tissue by a coagulation current. The radio-frequency voltage is usually applied to an electrode arrangement which is arranged on an inflatable balloon and which then results in a coagulation current via the body tissue.

An endoscope apparatus for coagulating body tissue is known from U.S. Pat. No. 6,904,303 B2 which has a balloon inflatable by a liquid and comprising a thermally conductive material and has an electrode inside the balloon for heating the liquid. The heat of the heated liquid is transmitted via the balloon to the body tissue to be treated. In another embodiment, the heated electrically conductive liquid as a conductor for radio frequency energy is transferred outwardly to the tissue through porous openings of the balloon.

An endoscope for coagulating body tissue is known from US 2007/028 7994 A1 in which a balloon is provided at the end which can be inflated by a liquid and which has a plurality of electrodes at the outside which are arranged lengthways and via which radio frequency energy is transmitted to the body tissue for bipolar treatment.

The body tissue is heated homogeneously in contact with the electrodes. This results in an areal coagulation. With non-homogeneously distributed or irregularly arranged electrode arrangements, the liquid is heated irregularly and non-homogeneously in the balloon. The upper portion of the balloon becomes hotter on a longer-lasting heating due to convection of the liquid in the balloon so that a non-homogeneous heat distribution in and around the balloon results.

Another known electrode arrangement for coagulating body cavities, e.g. a uterine cavity, has rigid preshaped electrodes. A further known electrode arrangement is used for coagulating bleeding gastrointestinal ulcers, with here small tips being used which are arranged over balloon catheters.

The positioning of a balloon in a small body cavity is difficult since it cannot be done in view. Thrombi occur around the balloon which provide non-reproducible conditions for the use of electrical fields or for radio frequency ablation (tissue denaturation), and indeed in comparison with the positioning of a defined balloon directly on the tissue to be treated.

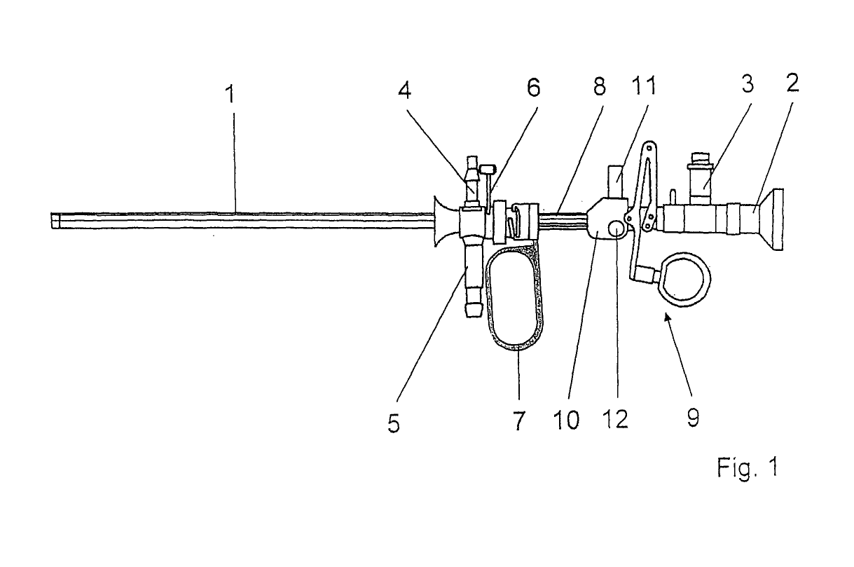

Medical instruments, in particular rigid endoscopes, for the resection of tissue are known. Different endoscopic resection instruments are thus described in chapter 1 of the text book “Endoskopische Urologie” [Endoscopic Urology] by Prof. Dr. R. Hofmann, 2nd Edition, 209, Springer Verlag. Such a known resectoscope is shown in FIG. 1. This known resectoscope comprises an instrument shaft 1, an endoscope optics 2 with a connector for a light cable 3 and a working element. The instrument shaft 1 usually has 24 charr. or 24 charr. circumference. The instrument shaft 1 can be used for continuous rinsing, i.e. a separate rinsing water inflow 4 and a sucking out 5 of the irrigation liquid (rinsing liquid) takes place. The water inflow is regulated by a faucet 6. Only the inflow of irrigation liquid can also take place which is let off with a full bladder by removing the working element from the instrument shaft 1.

The working element comprises a handle 7 for the third and fourth fingers. This handle 7 is fixedly attached to the instrument shaft 1 at a receiving tube 8 for the optics 2 and at a locking mechanism of the working element formed as a rotary closure. A moving restoring element 9 for the thumb grip is attached to the handle 7. A “passive resection instrument” is spoken of when a surgical noose formed as a surgical element is introduced into the instrument shaft 1 by the spring balance and is moved out against spring resistance. The working element moves passively back into the starting position again by spring force. An active working element is present when the surgical noose is moved out by the spring force in the relaxed state and a carriage 10 has to be retracted against the spring force using the thumb grip 9.

The movable carriage 10 is fastened to the thumb grip 9 and slides over the receiving tube 8 for the optics 2. It is usually an element which is made of plastic and which has an electrical feed line 11 as well as a button 12 for locking the electrode rod.

During endoscopic surgery, e.g. on a transurethral resection of a benign or also malignant prostate growth (TURP), tissue can be resected using a special cutting current from a radio frequency voltage generator either with a previously customary electrode arrangement or with a bipolar electrode arrangement. With the monopolar electrode arrangement, the current flows from an active electrode through a hypotonic, less conductive solution (e.g. a glycine solution, mannitol solution) through the body of the patient to a large areal neutral electrode. Two electrodes are located close to one another in the bipolar electrode arrangement. The current flows through a very conductive solution such as a saline solution from the one to the other electrode.

Bleeding regularly occurs during a TURP. Arterial and venous bleeding can now be directly atrophied in view by a coagulation current using the resection noose supplied with the radio frequency voltage. A spherical cavity is formed at the end of the TURP. Arterial bleeding has to be directly coagulated. With small arteries, a sintering of arterial bleeding is achieved by pressure using a balloon catheter. Diffuse venous bleeding from the whole resection area is usually present with a reduced or terminated rinsing flow at the end of the operation and it is only sintered by insertion of a balloon catheter.

A balloon can either be unfolded in the prostate cavity and then directly results in the compression of the venous bleeding or it is e.g. unfolded in the bladder located adjacent to the prostate cavity and is then pulled back to the prostate cavity so that hemostasis is achieved by compression of the prostate cavity.

The transurethral catheter usually has to be rinsed for some days and left to avoid a bladder tamponade. A substantial morbidity hereby arises for the patient such as pain in the urethra and in the bladder. Strangury, discharge from the urethra next to the catheter or urinary tract infections with fever occur. Protracted blood loss from the large wound area of the prostate cavity, due to the surgical procedure, or arterial bleeding, or more frequently venous bleeding, caused by friction and injury to the coagulation surface of the prostate cavity by the balloon are not rare.

In 2% to 5% of operations, postoperative urethral strictures occur after the TURP, with the longer indwelling time of an insert of a catheter in the urethra and an infection caused thereby being considered risk factors. The catheter frequently also clogs with blood thrombi if the continuous rinsing catheter is not regularly rinsed. The blood clots which form in this respect then have to be rinsed out of the catheter using bladder syringes. The catheter must also occasionally have to be replaced or a bladder tamponade has to be surgically excised under anesthetic.

A similar situation is present in suprapubic adenomectomy (removal of the prostate through the abdominal wall). Here, the large benign gland is enucleated by the surgeon either through the capsule of the prostate or transvesically (through the bladder) by the finger so that a large cavity arises. Diffuse venous bleeding usually remains which is stopped by transurethral insertion of a balloon catheter which is unfolded with pressure in the cavity. However, considerable complaints hereby arise for the patient for some days up to the catheter removal such as pain, foreign-body sensation in the rectum and in the urethra or bladder as well as stranguries due to the foreign body irritation.

It is therefore the object of the invention to provide an electrode arrangement of the initially named kind for coagulating body tissue and/or body vessels, wherein the coagulation can take place more simply, more effectively and faster for avoiding the named disadvantages of the known devices.

This object is satisfied by the features of claim 1. The ribbon-like electrode pairs can be brought to the positions to be treated simply and fast in their relaxed position. They can also be expanded in dependence on the available space of the body tissue and/or of the body vessels simultaneously and fast in touching contact with the body tissue and/or the body vessels. The correspondingly widely expanded electrode pairs then contact a large area at a desired pressure. A radio frequency control voltage of the radio frequency control voltage generator connected to all electrode pairs then generates a bipolar coagulation current simultaneously and over a large area in the body tissue. The coagulation effect is hereby amplified and the coagulation time shortened.

The electrode arrangement can be introduced through all commercial resection shafts in the relaxed state and can then be unfolded in endoscopic view and positioned in the prostate cavity. In the expanded state the electrode rows are expanded like meridians of a balloon. A coagulation flow respectively flows simultaneously between the two star-shaped electrode rows and effects an areal bipolar coagulation so that the venous bleeding or smaller arterial bleeding is stopped. The catheter indwelling time is shortened by a bleeding-free cavity at the end of surgery. The insertion of a transurethral catheter can therefore be dispensed with.

The coagulation takes place using a radio frequency control voltage generator suitable for radio frequency surgery. The control voltage has a frequency of approximately 900 to 600 kHz. The output power of the radio frequency control voltage generator amounts to at least 300 W. The coagulation takes place slowly so that no fast carbonization of the tissue takes place. It is advantageous if the radio frequency control voltage generator is adapted as a regulated generator to the impedance of the system with the two electrode groups and the body tissue. The SWR (standing wave ratio) can be measured before the coagulation process and can be fixed as the starting value. During the coagulation process, the impedance is then measured constantly and is kept in a previously defined range. The impedance of the system lies approximately between 10 and 15 ohms. The SWR increases during the coagulation. An exceeding of the upper limit value results in the switching off of the system. The operation time for the radio frequency control voltage generator is fixed by the surgeon. The SWR monitoring can, however, result in a premature automatic switching off.

The arrangement can be used in a similar manner for suprapbubic (by surgery from the abdominal wall) use, with now a handle with a sliding mechanism for folding and unfolding or expanding and relaxing the electrode arrangement, a locking arrangement for the electrode rod and a plug-in connection for the electrode cable becoming necessary.

Further advantageous embodiments of the invention result from the dependent claims.

The ribbon-like electrode pairs of the electrode arrangement can thus be expanded like meridians of a balloon.

If an operation is carried out by means of a surgical element, e.g. a resection noose, an endoscope introduced through an instrument shaft, this surgical element is removed after surgery and the electrode arrangement is introduced through the instrument shaft in its place without this instrument shaft having to be removed. It remains in the body. The time up to the start of the areal coagulation and the time for the coagulation itself are substantially shortened by this measure and by the simple and fast expanding of the electrode arrangement.

In particular the whole surgical instrument with the resection loop can thus in particular be swapped at the end of an operation. A coagulation apparatus with a relaxed electrode arrangement can then be introduced as a second working element into the resection shaft for the areal coagulation. A receiving tube for an endoscope optics is somewhat shorter than a usually utilized receiving tube which otherwise extends up to the end of the shaft. The receiving tube can be shortened so much that it ends shortly after a bayonet closure for the latching of the stabilization tube. The relaxed electrode arrangement can hereby also be introduced through small shaft diameters (e.g. 22 charr. or less). The coagulation apparatus is first introduced with the relaxed electrode arrangement through the instrument shaft and is latched with the shaft. It is thereby achieved that the electrode arrangement projects out of the shaft. Subsequently, the endoscope optics is introduced through the reception tube and the electrode arrangement is then expanded in view e.g. in the prostate cavity. The removal of the electrode arrangement takes place in reverse order, i.e. the electrode arrangement is first relaxed, then the endoscope optics is removed and subsequently the coagulation apparatus with the relaxed electrode arrangement is pulled out through the instrument shaft.

The electrode arrangement formed as an electrode rod can have an insulated infeed, a capillary draw tube and a hose sheath and can slide through a stabilization tube fixed to a receiving tube for the endoscope optics. A carriage can be moved actively or passively by moving to and fro, e.g. with the thumb, in a similar manner to a resection electrode. The first and second electrode groups can hereby be expanded, with the envelopes of the first and second electrode groups being axially displaced with respect to one another. The electrodes of these two electrode arrangements also have the same configuration in the expanded state.

In a first axial region, the envelope of the first electrode group lies within the envelope of the second electrode group. In an adjoining axial region, in contrast, the envelope of the first electrode group lies outside the envelope of the second electrode group. The electrode arrangement is positioned in the prostate cavity in view to avoid the electrodes of the electrode arrangement being incorrectly positioned. The electrode arrangement can hereby be ideally unfolded into the prostate cavity. The formation of thrombi during the areal coagulation is avoided by the simultaneous slow rinsing inflow. The electrodes firmly contact the tissue and adapt to irregularities due to their flexible design. The coagulation takes place in a saline solution between the respective adjacent electrodes of the first and second electrode groups which are held at the same spacing from one another in the expanded state.

An advantage of this electrode arrangement is that the electrode arrangement can be introduced through all commercial resection shafts in the relaxed state and can be positioned in endoscopic view e.g. in a prostate cavity, and can be expanded there. With the bipolar coagulation current between the individual expanded electrode pairs, a large-area coagulation is carried out by which a venous bleeding or smaller arterial bleeding can be stopped fast. The catheter indwelling time is shorted by a bleeding-free cavity at the end of surgery. An insertion of a transurethral catheter can be dispensed with.

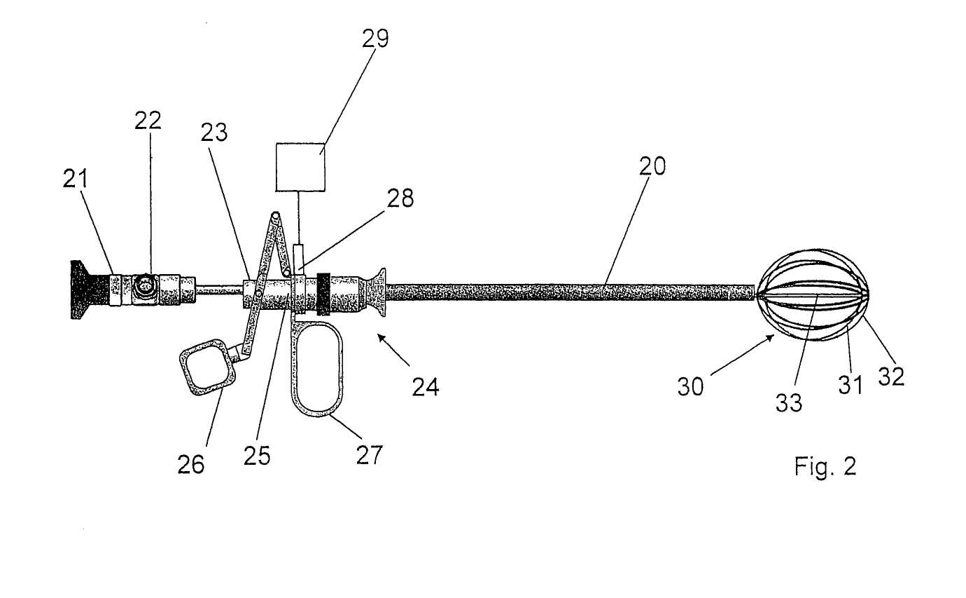

The invention will be described in the following with respect to the embodiments shown in FIGS. 2 to 10. There are shown:

FIG. 2 a schematic representation of an apparatus in accordance with the invention with an electrode arrangement expanded like meridians of a balloon;

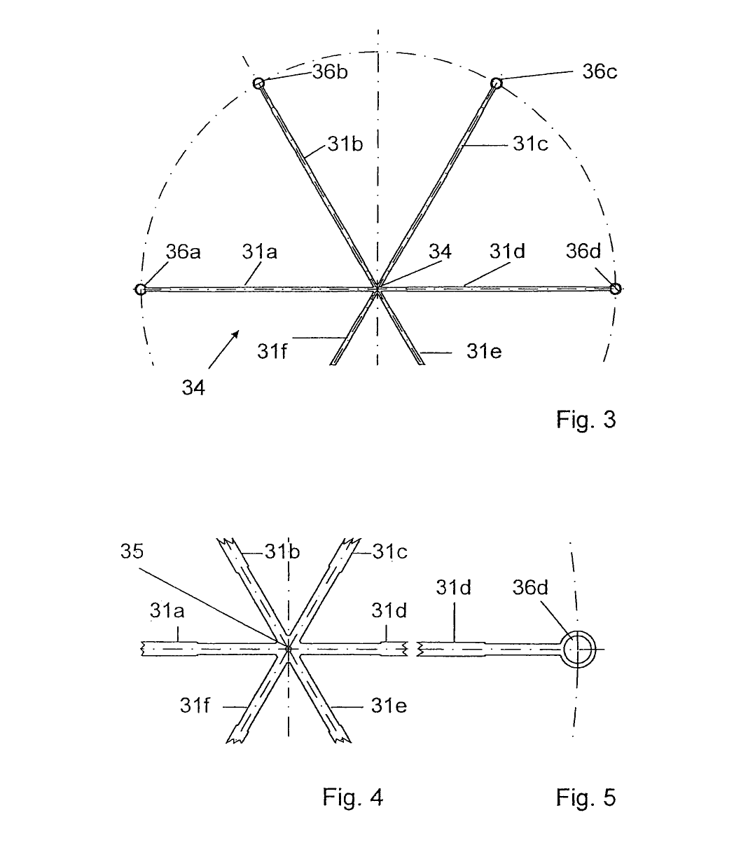

FIG. 3 the design of an electrode star of the first and second electrode groups;

FIG. 4 a detail of the central region (distal end) of the electrode star in accordance with FIG. 3;

FIG. 5 an oppositely disposed end (proximal end) of a ribbon-shaped electrode of the electrode start in accordance with FIG. 3;

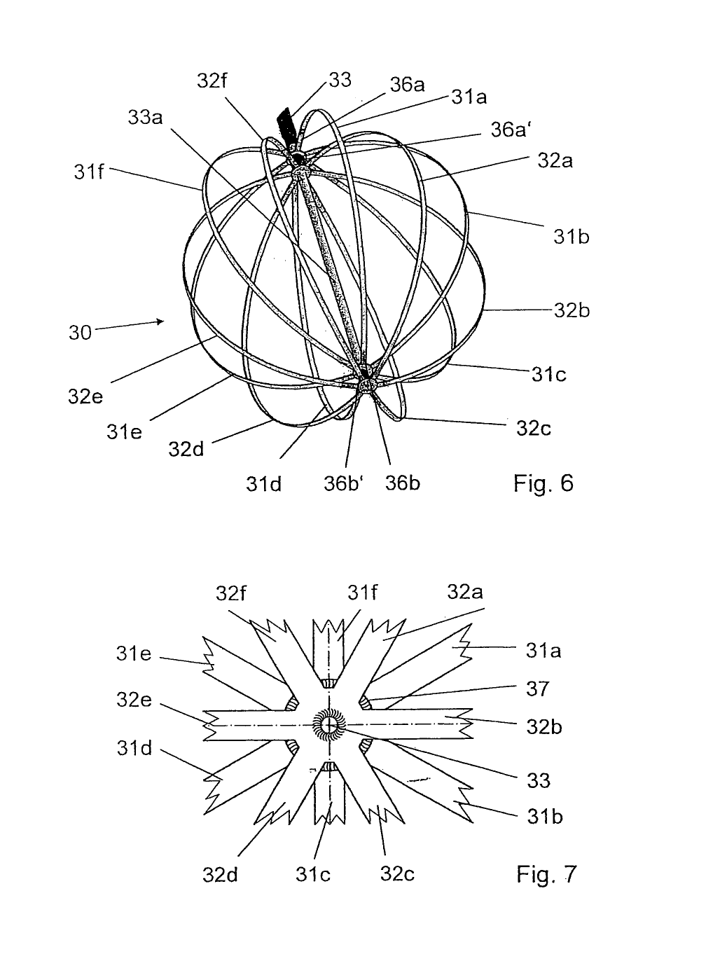

FIG. 6 a spatial representation of the electrode arrangement expanded like meridians of a balloon;

FIG. 7 a detail of the representation of FIG. 6;

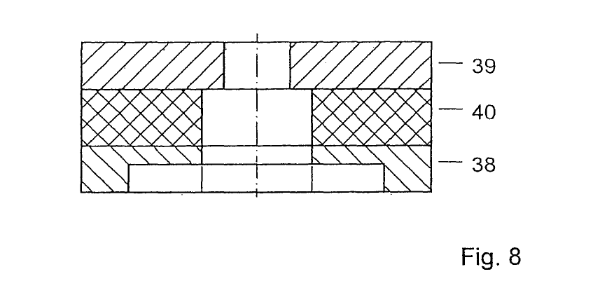

FIG. 8 an anode and cathode coupling at the distal end of the electrode arrangement with insulation plate;

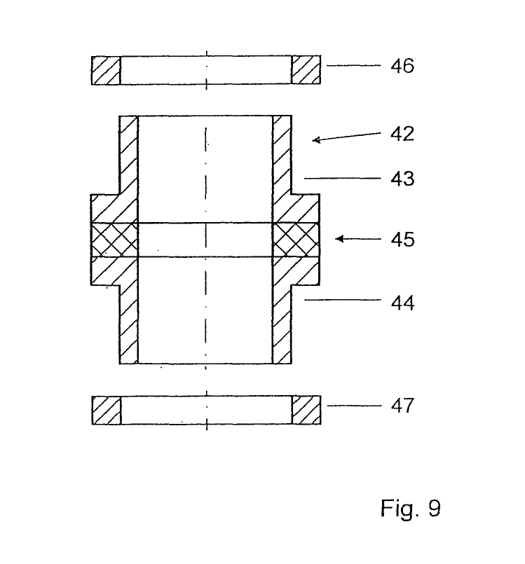

FIG. 9 a coupling plate with an insulating plate at the proximal end of the electrode arrangement; and

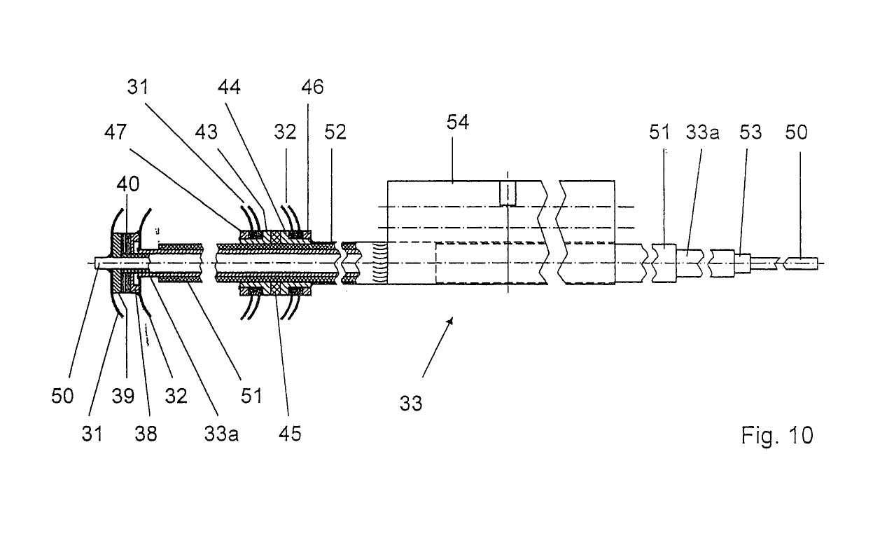

FIG. 10 an electrode rod in section.

In accordance with FIG. 2, a resectoscope includes an instrument shaft 20 and an endoscope optics 21 having a connector 22 for a light cable. A receiving tube 23 for the optics leads into an instrument housing 24. A movable carriage 25 is connected to a grip 26 actuable by a thumb. A handle for the third and fourth fingers is designated by 27. A radio frequency generator 29 which delivers a radio frequency coagulation voltage to an electrode arrangement in a manner not shown is connected to an anode connector and cathode connector 28. This electrode arrangement includes a first and second electrode group 31 and 32 which are insulated with respect to one another and which each form bipolar electrode pairs. The electrode pairs of the first and second electrode groups 31 and 32 are expanded in FIG. 2 like meridians of a balloon by a capillary draw tube 33a (FIG. 10) and an electrode rod 33 (FIG. 10), with the electrodes of the one electrode group 31 and of the other electrode group 32 alternating in the peripheral direction.

The endoscope optics 21 is first pulled out of the instrument shaft 20 to the left in a manner not shown with respect to FIG. 2 after an endoscopic operation. The surgical instrument, not shown, is subsequently removed through the instrument shaft 20 and is then replaced with the electrode rod 33 with electrode arrangement 30 which is likewise introduced from the left hand side, with in this respect the electrode groups 31 and 32 of the electrode arrangement 30 being relaxed and contacting the capillary draw tube 33a of the electrode rod 33. The endoscope optics 21 is then introduced again.

In FIG. 3, an electrode star 34 of the electrode group 31 is shown in the unwound (not installed) state. The individual electrodes or electrode arms 31a, 31b, 31c, 31d, 31e and 31f are in ribbon shape and flexible and are electrically conductive. In the embodiment shown, six electrodes 31a-31f are arranged in star shape and are connected to one another in one piece at a central region. The central region in accordance with FIG. 4 has a central opening 35 for an electric infeed 50 (FIG. 10). The outer ends 36a-36f of the electrode star 34 in FIG. 3, which are shown enlarged in FIG. 5, have openings to allow a passage of the capillary draw tube 33a of the electrode rod 33. The individual electrode arms, which are elastically deformable, are narrower towards their respective ends, as can in particular be seen from FIGS. 4 and 5, i.e. they have end sections with a reduced width. The coagulation effect is hereby reduced in this region.

Alternatively to this or also to optimize the reduction of the coagulation effect, the respective end regions can, in accordance with a further embodiment, be provided with insulation which extends, for example, over approximately the last 5-10 mm. It is hereby avoided that e.g. the apical (lower) region of the prostate with the sphincter disposed thereunder as well as the portion of the tissue extending into the bladder are heated too much on a coagulation.

The electrode arrangement 30 is shown in the expanded state in FIG. 6. The electrodes 31a-31f form the first electrode group 31, whereas electrodes 32a-32f form the second electrode group 32. The capillary draw tube 33a of the electrode rod 33 allows the axial displacement of the distal ends of the first and second electrode groups 31 and 32 into the position expanded to form the balloon. All the outer end regions 36a-36f or 36a′ to 36f of the individual electrodes 31a-31f and 32a to 32f are each provided with an insulating layer.

As in particular FIGS. 6 and 7 show, the first electrode group 31 is arranged axially rotated with respect to the second electrode group 32 in a manner such that the ribbon-shaped electrode arms 31a-31f of the first electrode group 31 each extend between the ribbon-shaped electrode arms 32a-32f of the second electrode group 32 and each form bipolar electrode pairs.

In a first (distal) region, the envelope of the first electrode group 31 lies within the envelope of the second electrode group 32. In an adjoining (proximal) axial region, in contrast, the envelope of the first electrode group 31 lies outside the envelope of the second electrode group 32, i.e. the two envelopes intersect (cf. also FIG. 2).

In FIG. 7, a detail of the proximal end region of the two electrode groups 31 and 32 is shown. The electrodes 31a-31f of the first electrode group 31 are arranged rotated about the center axis in the peripheral direction with respect to the electrodes 32a-32f so that the electrodes 31a, 32a-31, 32f each form electrode pairs. A coupling element shown enlarged in FIG. 9 is designated by 37.

In FIG. 8, an anode coupling 38 as well as a cathode coupling 39 are shown for the distal ends of the electrodes of the first and second electrode groups 31 and 32. A plate 40 insulating the two couplings are located between the anode coupling and the cathode coupling (cf. also FIG. 10).

A coupling sleeve 42 is shown in FIG. 9 which has two mutually insulated coupling sleeve parts 43 and 44 as well as an insulation 45 located therebetween. This coupling is provided at both sides with securing plates 46 and 47. In the installed state, the coupling sleeve 42 can slide axially over the electrode rod 33 so that a sliding mechanism can be reached.

This sliding mechanism is actuated by a movement of the hand as can be seen in FIG. 2, e.g. by a thumb movement. A locking screw, not shown, comprising a cylinder screw, a clamping screw and a knurled screw locks the capillary draw tube 33a of the electrode rod 33. The parts of the electrode rod 33 are moved with the aid of a carriage 25, not shown in any more detail, on a receiving tube 23 of the endoscope optics 21 so that the electrode groups 31 and 32 can thereby be expanded like the meridians of a balloon and can be relaxed again. The locking screw acts as a clamping element in the manner of a movable carriage which effects the coupling of the electrode.

FIG. 10 shows the electrode rod 33 in detail. It has a concentrically or coaxially arranged structure, with the electrical infeed 50 or the electrode cable which is insulated by an insulation 53, e.g. by Teflon, being arranged in the interior. The capillary draw tube 33a extends over this insulated line, extends up to the front end of the electrodes 31a-31f and 32a-32f of the two electrode groups 31 and 32 and is there connected to the coupling unit of FIG. 8.

An insulation hose 51, which likewise extends up to the front end, is located above the capillary draw tube 33a. The total arrangement is led through a stabilization tube 52 to whose distal end the coupling sleeve 42 (FIG. 9) is fastened.

The insulated electrical infeed 50 slides in the interior of the stabilization tube 52 which can be closed or also partly open longitudinally. The sliding mechanism allows the opening or closing or expanding and relaxing of the two electrode groups 31 and 32 by displacing the capillary draw tube 33a relative to the stabilization tube 52. The movement of the electrode rod 33 takes place such that the capillary draw tube 33a is fixed to a movable carriage 25 which is moved by the thumb grip of the working element. The capillary draw tube 33a which is provided with the insulation hose 51 thus slides in the stabilization tube 52 which is fixed to the receiving tube 8 for the optics 2. The electrode rod 33 is preferably fixed beneath the receiving tube 8 for the optics.

The stabilization tube 52 has a locking element, e.g. in the form of a bayonet closure 54, so that the total arrangement remains fixed to the working element. The receiving tube for the optics in the endoscope is connected by this bayonet closure to the stabilization tube 52 of the electrode rod 33.

An electrical plug-in connection for the electrode cable 50 is located at the carriage directly behind the locking element for the capillary draw tube 33a of the electrode rod 33.

The electrode arrangement is positioned in the prostate cavity in view to avoid the electrodes of the electrode arrangement being incorrectly positioned. The electrode arrangement can hereby be ideally unfolded into the prostate cavity. The formation of thrombi during the areal coagulation is avoided by the simultaneous slow rinsing inflow. The electrodes firmly contact the tissue and adapt to irregularities due to their flexible or elastic design.

1-18. (canceled)

19. An electrode arrangement for coagulating body tissues and/or body vessels, comprising
a first electrode group (31) and a second electrode group (32) which are insulated with respect to one another and which each have ribbon-shaped electrodes (31a-31f; 32a-32f) having proximal and distal ends, wherein the proximal ends are axially displaceable along an axis relative to the distal ends so that the electrodes of the first and second electrode groups (31a-31f; 32a-32f) can be expanded from a relaxed position into an expanded position to form a balloon; wherein
the first electrode group (31) is arranged axially offset with respect to the second electrode group (32) so that, in the expanded state of the electrodes, the envelope of the first electrode group (31) lies outside the envelope of the second electrode group (32) in a first axial region and inside the envelope of the second electrode group (32) in an adjoining second axial region.

20. An electrode arrangement in accordance with claim 19, wherein the first electrode group (31) is arranged rotated about the axis with respect to the second electrode group (32) in a manner such that the electrodes (31a-31f) of the first electrode group (31) extend in each case, viewed in the peripheral direction, between the electrodes (32a-32f) of the second electrode group (32).

21. An electrode arrangement in accordance with claim 19, wherein the electrodes of the first and second electrode group (31, 32) are provided in their respective end regions with an insulation (36a, 36b, 36a′, 36b′).

22. An electrode arrangement in accordance with claim 21, wherein the insulation (36a, 36b, 36a′, 36b′) extends over approximately the last 3-15 mm of the respective end regions.

23. An electrode arrangement in accordance with claim 19, wherein the electrodes (31a-31f; 32a-32f) can be expanded like meridians of a balloon.

24. An electrode arrangement in accordance with claim 19, wherein the distal ends are each held by a first central electrode receiving element (38, 39, 40); and wherein a second receiving element (42) axially displaceable relative to the first central electrode receiving element (38, 39, 40) is provided for receiving the proximal ends.

25. An electrode arrangement in accordance with claim 19, wherein the width of at least some electrodes reduces toward their respective ends.

26. An electrode arrangement in accordance with claim 19, wherein it has an electrode rod (33) which includes an inner insulated cable (50) which is led through a capillary draw tube (33a), with the capillary draw tube (33a) being sheathed outwardly by an insulating hose (51) which is led in a stabilization tube (52).

27. An electrode arrangement in accordance with claim 26, wherein the stabilizing tube (52) of the electrode rod (33) is movable over the hose (51) by a movement of the hand at a working element of an endoscope; and
wherein the capillary draw tube (33a) with the hose (51) and the insulated cable (50) are movable by the stabilization tube (52).

28. An electrode arrangement in accordance with claim 26, wherein the stabilization tube (52) can be fixed using a closure (54) at a receiving tube for an optics of an endoscope; and
wherein a carriage is provided in a sliding manner on the receiving tube which fixes the capillary draw tube (33a) by means of a clamping element so that by moving the carriage the electrode rod (33) is movable with respect to the stabilizing tube (52) for moving the electrode into the expanded position.

29. An electrode arrangement in accordance with claim 19, wherein the electrodes of the first and second electrode groups (31, 32) each form a first and second central region with their distal ends which is each held in an associated first electrode receiving element (38, 39, 40); and
wherein the proximal ends of the electrodes of the first and second electrode groups (31, 32) are held in a ring-shaped receiver (42).

30. An electrode arrangement in accordance with claim 19, wherein the distal ends of the electrodes of an electrode group (31) are connected to a cathode coupling (39) and the distal ends of the electrodes of the other electrode group (32) are connected to an anode coupling (38).

31. An electrode arrangement in accordance with claim 19, wherein the proximal ends of the electrodes of an electrode group (31) are connected to a cathode coupling sleeve (44) and the proximal ends of the electrodes of the other electrode group (32) are connected to an anode coupling sleeve (43) insulated with respect to the cathode coupling sleeve (44).

32. An electrode arrangement in accordance with claim 31, wherein the cathode coupling sleeve (44) and the anode coupling sleeve (43) are secured by securing plates (45, 47).

33. An electrode arrangement in accordance with claim 19, wherein it is connected to an endoscope, such that the electrodes of the first and second electrode groups (31, 32) can be pushed into an instrument shaft (20) in the relaxed position and can then be latched thereto.

34. An electrode arrangement in accordance with claim 19, wherein it is connected to a rectoscope, such that the electrodes of the first and second electrode groups (31, 32) can be pushed into an instrument shaft (20) in the relaxed position and can then be latched thereto.

35. A method of coagulating body tissue and/or body vessels, wherein
a plurality of ribbon-shaped electrode pairs (31a-31f; 32a-32f) for touching contact with the body tissue and/or the body vessels are expanded to form a balloon; and
a radio frequency control voltage is applied to the electrode pairs (31a-31f; 32a-32f) for generating a regulated, large-area bipolar coagulation current between the electrodes of the respective electrode pairs (31a-31f; 32a-32f).

36. A method in accordance with claim 35, wherein an electrode arrangement comprising:
a first electrode group (31) and a second electrode group (32) which are insulated with respect to one another and which each have ribbon-shaped electrodes (31a-31f; 32a-32f) having proximal and distal ends, wherein the proximal ends are axially displaceable along an axis relative to the distal ends so that the electrodes of the first and second electrode groups (31a-31f; 32a-32f) can be expanded from a relaxed position into an expanded position to form a balloon; wherein the first electrode group (31) is arranged axially offset with respect to the second electrode group (32) so that, in the expanded state of the electrodes, the envelope of the first electrode group (31) lies outside the envelope of the second electrode group (32) in a first axial region and inside the envelope of the second electrode group (32) in an adjoining second axial region, is used.

37. A method in accordance with claim 35, wherein the electrode pairs are expanded like meridians of a balloon.

38. A method in accordance with claim 35, wherein the electrode arrangement is introduced into an instrument shaft of an endoscope after previously removing a surgical apparatus from the instrument shaft.

39. A method in accordance with claim 38, wherein subsequently an endoscope optics (21) is attached to the endoscope.
